(19) United States
(12) Patent Application Publication
(10) Pub. No.: US 2008/0057347 A1
(43) Pub. Date: Mar. 6, 2008

(54) ULTRAVIOLET-FLOURESCING MATERIAL

(76) Inventors: David G. Foster, West Henrietta, NY (US); Maurice L. Gray, Rochester, NY (US); William H. Simpson, Pittsford, NY (US)

Correspondence Address: EASTMAN KODAK COMPANY;PATENT LEGAL STAFF, 343 STATE STREET, ROCHESTER, NY, 14650-2201, US

(21) Appl. No.: 11/928,190

(22) Filed: Oct. 30, 2007

Related U.S. Application Data
(63) Continuation of application No. 10/972,716, filed on Oct. 25, 2004, now abandoned.

(51) Int. Cl. B32B 7/00; B05D 3/00; C07D 265/22

(52) U.S. Cl. 428/704; 252/589; 427/160; 544/92

(57) ABSTRACT

An ultraviolet absorbing, fluorescing material is described, wherein the material fluoresces and can provide ultraviolet protection.

### CROSS-REFERENCE TO RELATED APPLICATIONS

This is a continuation of application Ser. No. 10/972,716, filed Oct. 25, 2004.

### FIELD OF THE INVENTION

This invention relates to an ultraviolet absorbing, fluorescing material that fluoresces and provides ultraviolet protection.

### BACKGROUND OF THE INVENTION

Thermal transfer systems have been developed to obtain prints from pictures that have been generated electronically, for example, from a color video camera or digital camera. An electronic picture can be subjected to color separation by color filters. The respective color-separated images can be converted into electrical signals. These signals can be operated on to produce cyan, magenta, and yellow electrical signals. These signals can be transmitted to a thermal printer. To obtain a print, a black, cyan, magenta, or yellow dye-donor layer, for example, can be placed face-to-face with a dye image-receiving layer of a receiver element to form a print assembly which can be inserted between a thermal print head and a platen roller. A thermal print head can be used to apply heat from the back of the dye-donor sheet. The thermal print head can be heated up sequentially in response to the black, cyan, magenta, or yellow signals. The process can be repeated as needed to print all colors. A color hard copy corresponding to the original picture can be obtained. Further details of this process and an apparatus for carrying it out are contained in U.S. Pat. No. 4,621,271 to Brownstein.

Thermal prints are susceptible to retransfer of dyes to adjacent surfaces and to discoloration by fingerprints. This is due to dye being at the surface of the dye-receiving layer of the print. These dyes can be driven further into the dye-receiving layer by thermally fusing the print with either hot rollers or a thermal head. This will help to reduce dye retransfer and fingerprint susceptibility, but does not eliminate these problems. The application of a protection overcoat can reduce or eliminate the occurrence of these problems. The protection overcoat can be applied to the dyed receiver element by heating the donor element to transfer the protective overcoat. The protection overcoat can improve the stability of the image to light fade and oil from fingerprints.

Exposure of dyes to ultraviolet (UV) light, that is light with wavelengths less than 400 nm, usually results in degradation, or fading, of dyes with time. The degradation can be caused by photolysis, which is the direct absorption of the ultraviolet light. The dye can also degrade by either photooxidation or photoreduction depending on the chemical structure of the dye and of the natural or man-made polymer surrounding the dye. It is common to include a UV-absorbing material in a heat-transferable protective overcoat layer for a dye-diffusion thermal transfer print as taught in U.S. Pat. No. 4,522,881 to reduce the rate of dye fading from ultraviolet light. U.S. Pat. NO. 6,184,375 and U.S. Patent Application Publication US 2003/0176283A1, filed 18 Mar. 2002, suggest the use of triazine UV absorbers for a broad range of uses. It is suggested to employ such materials in various locations such as the receiver layer or in the overcoat, as described in EP 505,734.

UV-absorbers can also be used in other colored formulations, for example, ink jet inks, paints, colorants, and dyes, to prevent colorant fade, wherein “colorant” refers to pigment, dye, or a combination thereof. UV-absorbers can be used in the printing and graphic arts to prevent colorant fade. For example, EP0407615B1 describes a heat transfer recording medium with a transparent protective layer containing a transparent pigment, a dye, a fluorescent dye, an infrared absorber, a fluorescent brightener, an ultraviolet absorber, an oxygen indicator, or a hologram, where at least part of the surface of the protective layer functions to provide security against counterfeiting. EP0982149B1 describes a fluorescent latent image transfer film using a security pattern. EP1346839A2 describes a security element with a fluorescent colorant layer provided on the substrate.

Colorants can be used for many different applications. For example, in printing or coating arts, colorants can be applied to various media, including identification (ID) cards, credit or bank cards, membership cards, tickets, gift items, novelties, clothing, jewelry, paper-like materials, printing receivers, and photographic receivers. It is beneficial to have UW protection in such items. However, it can also be desirable to provide other features, such as fluorescence, on such items for artistic or security purposes.

The use of fluorescing material as a security feature in various media, for example, tickets, credit cards, handstamps, and driver's licenses, is known. The fluorescing material can be used to form a pattern, word, number, or symbol visible under certain wavelengths of light. The purpose of the fluorescing material is to ensure the security-marked item is an original, and that no tampering has occurred.

It would be desirable to combine the functions of UV-protection and fluorescence in a single material for use in a composition to reduce manufacturing costs and time, and to provide one or more of UV-protection, aesthetic detail, and a security feature.

### SUMMARY OF THE INVENTION

A composition comprising an ultraviolet absorbing, fluorescing material, and uses thereof are described.

### DETAILED DESCRIPTION OF THE INVENTION

An ultraviolet absorbing, fluorescing material for use in a composition is described, wherein the material can function as both a UV-absorber and a fluorescing material.

The material can absorb UV light at wavelengths less than 400 nm.

The material can have a maximum absorption at a wavelength less than 400 nm. The material can be non-light-absorbing from 400 to 800 nm.

Peak fluorescence emission of the material can be in the range of from 300 to 900 nm, for example, in the range of from 400-800 nm.

Examples of ultraviolet absorbing, fluorescing materials that absorb UV light and fluoresce can include the following structure: 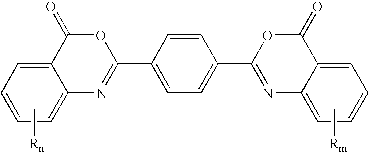
wherein each R can independently be selected from hydrogen or a C₁₋₆ straight chain alkyl, and m and n can each independently be chosen from 0-4. For example, the material can be 2,2′-(1,4-phenylnen)bis[4H-3,1-benzoxazin-4-one], which can be obtained as Cyasorb® UV-3638 from Cytec Industries, Inc., West Paterson, N.J., and has the following structure:

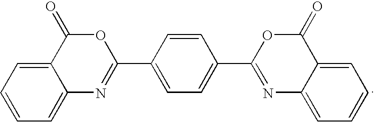

The ultraviolet absorbing, fluorescing material can be present in a composition in an amount of at least 0.001 g/m². For example, the ultraviolet absorbing, fluorescing material can be present in an amount of 0.04 g/m² to 2.0 g/m², for example 0.07 g/m² to 0.80 g/m². The ultraviolet absorbing, fluorescing material can be present in an amount that is sufficient to provide UV-protection, fluorescence, or both.

The ultraviolet absorbing, fluorescing material can be used in various compositions. For example, the ultraviolet absorbing, fluorescing material can be used in a composition containing a dye, pigment, or colorant. The composition can be, for example, an ink jet ink, thermal dye-donor layer, laser thermal-dye transfer layer, an ink composition useful in graphic arts, a paint a colorant, a wash, or an adhesive. As used herein, a colorant or ink can contain one or more pigment, one or more dye, or a combination thereof Dyes and pigments can include visible, fluorescent, and UV-absorbing dyes and pigments.

The ultraviolet absorbing, fluorescing material can be in various coating compositions, for example, overcoats for ink jet ink, thermal dye transfer, or laser thermal dye transfer images; laminates; or protective plastic covers.

The ultraviolet absorbing, fluorescing material can be dispersed in a composition, for example, a colorant-containing composition, or a polymeric composition. The ultraviolet absorbing, fluorescing material can be dispersed or mixed in a binder, a solvent, or a combination thereof, and applied to a material by any known means, such as coating, printing, brushing, or spraying.

Suitable binders can include polymeric binders, for example, a polycarbonate; a poly(styrene-co-acrylonitrile); a poly(sulfone); a poly(phenylene oxide); a cellulose derivative such as but not limited to cellulose acetate hydrogen phthalate, cellulose acetate, cellulose acetate propionate, cellulose acetate butyrate, or cellulose triacetate; or a combination thereof For example, suitable binders for use in a protective overcoat or laminate can include, but are not limited to, the following:

1) Poly(vinyl benzal) in 2-butanone solvent;

2) Poly(vinyl acetal) KS-5 (Sekisui Co) (26 mole % hydroxyl, 74 mole % acetal) in a 3-pentanone/methanol solvent mixture (75/25);

3) Poly(vinyl acetal) KS-3 (Sekisui Co) (12 mole % hydroxyl, 4 mole % acetate, 84 mole % acetal) in a 3-pentanone/methanol solvent mixture (75/25);

4) Poly(vinyl acetal) KS-1 (Sekisui Co) (24 mole % hydroxyl, 76 mole % acetal) in a 3-pentanone/methanol solvent mixture (75/25);

5) Poly(vinyl acetal) (26 mole % hydroxyl, 74 mole % acetal) in a 3-pentanone/methanol solvent mixture (75/25);

6) Poly(vinyl acetal) (29 mole % hydroxyl, 71 mole % acetal) in a 3-pentanone/methanol solvent mixture (75/25);

7) Poly(vinyl acetal) (56 mole % hydroxyl, 44 mole % acetal) in a 3-pentanone/methanol solvent mixture (75/25);

8) Poly(vinyl acetal) (15 mole % hydroxyl, 77 mole % acetal, 8 mole % acetate) in a methanol/3-pentanone solvent mixture (75/25);

9) Poly(vinyl acetal) (20 mole % hydroxyl 51 mole % acetal, 29 mole % acetate) in a methanol/3-pentanone solvent mixture (75/25);

10) Poly(vinyl acetal) (24 mole % hydroxyl, 76 mole % acetal) in a methanol/3-pentanone solvent mixture (75/25);

11) Poly(vinyl acetal) (44 mole % hydroxyl,. 43 mole % acetal, 13 mole % acetate) in a methanol/water solvent mixture (75/25);

12) Poly(vinyl acetal) (65 mole % hydroxyl, 35 mole % acetal) in a methanol/water solvent mixture (75/25);

13) Poly(vinyl acetal) (18 mole % hydroxyl, 64 mole % acetal, 18 mole % acetate) in a methanol/3-pentanone solvent mixture (75/25);

14) Poly(vinyl acetal) (16 mole % hydroxyl, 84 mole % acetal) in a methanol/3-pentanone solvent mixture (75/25); and

15) Poly(vinyl formal) (Fomnvar®, Monsanto Co.) (5% hydroxyl, 82% formal, 13% acetate) in a toluene/3A alcohol/water mixture (57/40/3).

The ultraviolet absorbing, fluorescing material can be applied to or used in a layer of a composition, for example, an identification card, credit card, bank card, membership card, ticket, gift item, novelty, article of clothing, jewelry, natural paper, synthetic paper, printing receiver, or photographic receiver. The ultraviolet absorbing, fluorescing material can also be used in various colorant compositions for direct or indirect application to a material, for example, paper, cloth, plastic, skin, metal or glass.

The ultraviolet absorbing, fluorescing material, or a composition containing it, can be applied to a receptive material in an image-wise pattern directly or through an intermediate transfer process. The pattern can be the same or different for each transferred area. For example, for security, the ultraviolet absorbing, fluorescing material can be transferred in a pattern, for example, stripes, a checkerboard, or waves; a symbol; a company insignia or logo; a picture; a landscape; a word; a number; other indicia; or a combination thereof. The ultraviolet absorbing, fluorescing material or composition containing it can also be applied as a uniform or non-uniform layer over all or a portion of the surface of a receiver. A non-uniform layer can include higher and lower density areas of the ultraviolet absorbing, fluorescing material, forming a random pattern or an intentional pattern or image. When the ultraviolet absorbing, fluorescing material is transferred, the transferred material preferably does not totally obscure any image, text, or pattern preexisting on the receptive material. When the ultraviolet absorbing, fluorescing material is made to fluoresce, it can obscure an image or pattern beneath the material, or any image or pattern can remain at least partially discernable beneath the ultraviolet absorbing, fluorescing material.

Receptive materials can include any surface capable of being coated with a ultraviolet absorbing, fluorescing material, and can include, but are not limited to, an identification card, credit card, bank card, membership card, ticket, gift item, novelty, article of clothing, jewelry, natural paper, synthetic paper, printing receiver, photographic receiver, cloth, plastic, skin, metal or glass.

According to certain embodiments, the receptive material can be printed with a hologram, photograph, logo, alphanumeric, pattern, or other image. The ultraviolet absorbing, fluorescing material can be overlaid on the image as a uniform layer or pattern. The ultraviolet absorbing, fluorescing material can also be placed under an image, or interspersed with colorants forming an image on a receptive material.

Use of the ultraviolet absorbing, fluorescing material enables UV protection of colorants in the receptive material, while also adding a fluorescing aspect for security or decorative purposes.

### EXAMPLES

### Example 1

A neutral density image with a maximum density of at least 2.3 was printed using a Kodak 8650 Thermal Printer on a receiver of Kodak Ektatherm®, Catalog #172-5514, using the donor elements described below.

To form the image, a dye donor element having a protection layer was placed in contact with the polymeric receiving layer side of the receiver to print areas of neutral, red, green, and blue density, with a laminate overcoat. The assemblage of donor and receiver was positioned on an 18 mm platen roller and a TDK thermal head (No. 3K0345) with a head load of 62 Newtons was pressed against the platen roller. The TDK 3K0345 thermal print head has a resolution of 300 dots/inch and an average resistance of 3314 Ω. The imaging electronics were activated when an initial print head temperature of 36.4° C. was reached. The assemblage was drawn between the printing head and platen roller at 16.9 mm/sec.

The resistive elements in the thermal print head were pulsed on for 58 μsec every 76 μsec. 64 pulses “on” time were applied per line, with as line time of 5.0 milliseconds. The voltage supplied at 13.6 volts resulted in an instantaneous peak power of approximately 58.18×10⁻³ Watt/dot and the maximum total energy required to print Dmax was 0.216 mJoules/dot.

This printing process heated the laminate uniformly with the thermal head to permanently adhere the laminate to the colored print. The donor support was peeled away as the printer advanced through its heating cycle, leaving the laminate adhered to the imaged receiver.

Comparative Donor Element C-1:

A transferable overcoat was prepared by coating the following 10 layers in the order recited on 6.0 micron poly(ethylene terephthalate) support:
(1) a subbing layer of a titanium alkoxide (duPont Tyzor TBT®) (0.12 g/m²) from n-propyl acetate and n-butyl alcohol solvent mixture (85/15), and
(2) a transferable protective overcoat layer of poly(vinyl acetal), KS-10, (Sekisui Co.), at a laydown of 0.63 g/m²; colloidal silica, IPA-ST (Nissan Chemical Co.), at a laydown of 0.46 g/m²; and 4 μm divinylbenzene beads at a laydown of 0.11 g/m². The materials were coated from a 78.9/21.1 solvent mixture of diethyl ketone and isopropanol.

A dye donor was prepared by coating the above transferable overcoat on a 6.0 micron poly(ethylene terephthalate) support, and coating on the opposite side of the support the following slipping layer in the order recited:
(1) a subbing layer of a titanium alkoxide (duPont Tyzor TBT®) (0.1 2 g/m2) from n-propyl acetate and n-butyl alcohol solvent mixture (85/15), and
(2) a slipping layer containing an aminopropyl-dimethyl-terminated polydimethylsiloxane, PS513® (United Chemical Technologies) (0.01 g/m²); a poly(vinylacetal) binder (0.38g/m²) (Sekisui KS-1); p-toluenesulfonic acid (0.0003 g/m²); and candellila wax (0.02 g/m²) coated from a 88.7/9.0/2.3 solvent mixture of diethylketone, methanol and distilled water.
   Inventive Donor Elements I-1 to 7:

These donor elements are the same as C-1 with the addition to the transferable overcoat layer of Cyasorb UV3638 (2,2′-(1,4-Phenylene)bis[4H-3,1 -benzoxazin-4-one], CAS Number 018600-594), at a lay down of 0.0634, 0.0314, 0.0157, 0.0078, 0.0039, 0.0020 and 0.0010 g/m², respectively.

Test Procedure:

To test for the fluorescing nature of these elements, the donors and printed receivers were exposed to a UV lamp (Mineralight, UVS-11, 115 volts, 60 cycles, 1.2 amps, from Ultra-Violet Products, Inc., San Gabriel, Calif.). Donor elements as well as prints made from them were placed on a standard laboratory bench-top in the dark and the UV lamp was shown on each in turn to examine whether or not the donor elements or the printed receiver fluoresced. Table 1 below illustrates the results of this analysis.

TABLE 1

| | | | Does the | Does the Print Made |
|---|---|---|---|---|
| | Organic | Coverage | Coating | from the Coating |
| Example ID | Material | (g/m²) | Fluoresce? | Fluoresce? |
| Control C-1 | None | None | No | No |
| Invention I-1 | UV-3638 | 0.0634 | Yes | Yes |
| Invention I-2 | UV-3638 | 0.0314 | Yes | Yes |
| Invention I-3 | UV-3638 | 0.0157 | Yes | Yes |
| Invention I-4 | UV-3638 | 0.0078 | Yes | Yes |
| Invention I-5 | UV-3638 | 0.0039 | Yes | Yes |
| Invention I-6 | UV-3638 | 0.0020 | Yes | Yes |
| Invention I-7 | UV-3638 | 0.0010 | Yes | Yes |

The results in Table 1 indicate that incorporating UV-3638 material into the over-protective laminate formulation creates a fluorescing coating on the donor element that can be transferred to give a fluorescing overcoat on the receiver.

### Example 2

A donor element the same as C-1 was used with the addition to the transferable overcoat layer of Cyasorb UV3638 (2,2′-(1,4-Phenylene)bis[4H-3,1-benzoxazin-4-one], CAS Number 018600-59-4), at a laydown of 0.4180 g/m².

The usefulness of UV-3638 as an ultraviolet (UV) screening agent in a protection overcoat was examined using samples which were printed on a receiver as described in Example 1, and then subjected to light in a high intensity 50 klux sunlight unit for 14 days to examine light fade. The sunlight unit has a xenon light source and no UV filter. After exposure, the samples were removed from the light chamber and density readings were made and compared to pre-exposure densities, providing a density loss for the control and inventive samples. The results included in Table 2 are the difference in percentage change in density corresponding to the density lost in the control minus the density lost in the inventive sample for each of neutral, red, green, and blue areas of the printed receiver. The larger the number, the more dye lost in the control relative to the invention. Dye loss can be from UV degradation.

TABLE 2

| Colored | | | |
|---|---|---|---|
| Printed | Red | Green | Blue |
| Areas | Delta | Delta | Delta |
| Neutral | 9.12 | 17.14 | 46.74 |
| Green | 10.78 | | 46.23 |
| Red | | 28.74 | 50.28 |
| Blue | 9.31 | 18.11 | |

The results in Table 2 indicate that incorporating UV-3638 material into the protective overcoat decreases the amount of dye density lost due to UV damage relative to the control.

### Example 3

An inkjet ink made with UV-fluorescent material as follows. Ink was removed from a conventional black inkjet cartridge and to this ink was added 0.1 g/L of UV3638. The ink was replaced in the cartridge and then a page of conventional text was printed. After printing, the page was taken and exposed to a UV lamp. The print on the page was observed to fluoresce.

### Example 4

The ability of UV3638 to fluoresce when applied to different surfaces and receivers was tested by dissolving 0.1 g/L of UV3638 in 3-pentanone. The mixture was placed in a common handheld spray bottle of the type used to dispense glass cleaner and other cleaning products. Numerous materials were then sprayed with the mixture and the UV lamp was shown on the sprayed materials to observe whether or not fluorescence occurred. Table 3 below illustrates the results.

TABLE 3

| | Fluorescence |
|---|---|
| Material | Observed? |
| Metal wall | Yes |
| Fabric covered surface | Yes |
| Paper towel | Yes |
| White Paper | Yes |
| Glass | Yes |
| Human Skin | Yes |

As the table illustrates, all materials tested fluoresced when treated with UV3638 and exposed to ultraviolet light. It is anticipated that any material to which the ultraviolet absorbing, fluorescing material described herein is applied will exhibit a fluorescing nature.

The invention has been described in detail with particular reference to certain preferred embodiments thereof but it will be understood that variations and modifications can be effected within the spirit and scope of the invention.

1. A dye-donor element comprising a substrate, a dye-donor layer, and an overcoat, wherein the dye-donor element comprises a ultraviolet absorbing, fluorescing material.

2. The dye-donor element of claim 1, wherein the ultraviolet absorbing, fluorescing material is in the overcoat, the dye-donor layer, or both.

3. The dye-donor element of claim 1, wherein the UV- fluorescing material has the following structure: 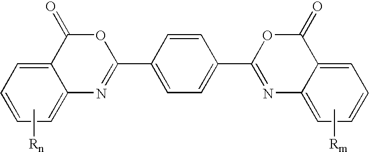
wherein each R can independently be selected from hydrogen or a C₁₋₆ straight chain alkyl, and m and n can each independently be chosen from 0-4.

4. The dye-donor element of claim 1, wherein said ultraviolet absorbing, fluorescing material is not visually observable under visible light.

5. A receptive element having a layer comprising a UV-fluorescing material.

6. The receiver of claim 5, wherein the UV-fluorescing material has the following structure: 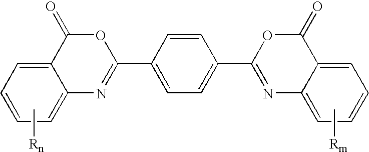
wherein each R can independently be selected from hydrogen or a C₁₋₆ straight chain alkyl, and m and n can each independently be chosen from 0-4.

7. The receiver of claim 5, wherein said ultraviolet absorbing, fluorescing material is not visually observable under visible light.

8. The receiver of claim 5, wherein the layer is a protective layer.

9. The receiver of claim 5, wherein the layer further comprises a dye, pigment, or combination thereof.

10. The receiver of claim 5, wherein the ultraviolet absorbing, fluorescing material is in an amount of at least 0.001 g/m².

11. The receiver of claim 5, wherein the ultraviolet absorbing, fluorescing material is in an amount of 0.04 to 2.0 g/m².

12. The receiver of claim 5, further comprising a layer including a UV-absorbing non-fluorescing material, fluorescing material, or both.

13. The receiver of claim 5, wherein the receiver is an identification card, credit card, bank card, membership card, ticket, gift item, novelty, article of clothing, jewelry, natural paper, synthetic paper, printing receiver, photographic receiver, plastic, glass, or skin.

14. The receiver of claim 5, wherein the ultraviolet absorbing, fluorescing material forms an image, a pattern, words, numbers, indicia, or a combination thereof.

15. A print assembly comprising the dye-donor element of claim 11 and a receiver.

16. A method of forming an image, comprising:
obtaining a composition including a ultraviolet absorbing, fluorescing material;
obtaining a receptive material;
applying the composition to the receptive material.

17. The method of claim 16, wherein the composition is applied to the receptive material in an image-wise fashion.

18. The method of claim 16, wherein the composition is applied to an intermediate material, and then transferred from the intermediate material to the receptive material.

19. The method of claim 16, wherein the image is formed by printing, coating, or a combination thereof.

20. The method of claim 16, wherein the receptive material is an identification card, credit card, bank card, membership card, ticket, gift item, novelty, article of clothing, jewelry, natural paper, synthetic paper, printing receiver, photographic receiver, plastic, glass, or skin.
